(19) United States
(12) Patent Application Publication
(10) Pub. No.: US 2010/0022943 A1
(43) Pub. Date: Jan. 28, 2010

(54) Hydrodynamic Thrombectomy Catheter

(75) Inventors: Kevin Mauch, Windsor, CA (US); Maria Arreguin, Windsor, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/180,122

(22) Filed: Jul. 25, 2008

(51) Int. Cl. A61B 17/32; A61M 25/00

(52) U.S. Cl. 604/22; 604/508

(57) ABSTRACT

A catheter apparatus for removing an obstruction within a body lumen includes an elongate tubular shaft defining a lumen and a flexible membrane that fluidly seals the distal end of the tubular shaft. At least one cutter member is attached to and distally extends from the flexible membrane. An actuating mechanism is operatively connected to a proximal end of the tubular shaft. The actuating mechanism displaces a fluid disposed within the lumen of the tubular shaft in such a manner that the fluid oscillates the flexible membrane and the cutter member attached thereto. Accordingly, the catheter apparatus uses pulsatile fluid flow through the tubular shaft to transmit energy from the driving mechanical at the proximal end of the catheter apparatus to the flexible membrane at the distal end of the catheter apparatus. The transmitted energy causes the cutting member to oscillate and break up a target blood clot.

### FIELD OF THE INVENTION

The invention relates to a hydrodynamic thrombectomy catheter device for percutaneous removal of clots or obstructions within the vascular system.

### BACKGROUND OF THE INVENTION

Human blood vessels may become occluded or completely blocked by thrombi (blood clots), which reduce the blood carrying capacity of the vessel. Some disease states associated with blood clots include deep vein thrombosis, stroke, and acute myocardial infarction. Blood clots may appear in the brain, veins, lungs, heart, or arteries. If the blockage occurs at a critical place in the circulatory system, serious and permanent injury, or even death, can occur.

To prevent such adverse consequences, some form of medical intervention is usually performed when significant occlusion is detected. Thrombectomy is a term used to refer to a technique that breaks up or removes a blood clot to allow increased blood flow through the vessel. One technique to remove a blood clot includes infusing a thrombolytic agent to dissolve the clot. Another technique to remove a blood clot utilizes a Fogarty catheter that passes a balloon through the clot, expands the balloon, and then pulls the balloon proximally to engage and subsequently remove the clot. Some retrieval devices include corkscrew or snare retrieval elements for engaging and subsequently removing a blood clot.

Other retrieval devices include energy based systems, such as the use of water jets, laser, or ultrasound energy, to break up the clot. Such devices may additionally include mechanical means at the distal end of the device to break up the clot such as mechanical cutters, augers, and vibrating wires. These energy and mechanical based systems require a drive shaft running through a lumen of the catheter in order to allow the transfer of energy from the proximal end to the distal end of the device. The drive shaft causes a relatively large profile catheter, and also causes the catheter to be stiff and inflexible and have limited applicability. For example, such thrombectomy devices may not be used for removing blood clots from the intracranial circulation since the blood vessels in the brain are very small and tortuous. In addition, the motors of these devices may be susceptible to stalling out due to friction loss when the catheter is snaked through a tortuous vessel. Thus, there remains a need in the art for an improved device to remove thrombi and emboli.

### BRIEF SUMMARY OF THE INVENTION

Embodiments of the present invention relate to a catheter apparatus for removing an obstruction within a body lumen. The catheter apparatus includes an elongate tubular shaft defining a lumen with a sealed or closed distal end such that a volume of fluid is contained therein. A shape memory wire member is attached to the tubular shaft, the wire member having a bend occurring along a distal length thereof. A distal end of the wire member extends distally beyond a distal end of the tubular shaft to define a cutting element. An actuating mechanism is operatively connected to a proximal end of the tubular shaft that applies a sufficient force to displace the fluid disposed within the lumen of the tubular shaft to straighten the tubular shaft and the wire member. The cutting member oscillates as the wire member alternates between bent and straightened configurations when the actuating mechanism applies and removes the force in rapid succession.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a schematic sectional view illustration of a hydrodynamic thrombectomy catheter according to an embodiment of the present invention, wherein the catheter includes a distally extending cutting element.

FIG. 2 is a schematic sectional view illustration of a hydrodynamic thrombectomy catheter according to another embodiment of the present invention, wherein the catheter includes an aspiration lumen.

FIG. 3 is a schematic side view illustration of a distal portion of a hydrodynamic thrombectomy catheter having a distally extending cutting element according to another embodiment of the present invention.

FIG. 4 is a schematic side view illustration of a distal portion of a hydrodynamic thrombectomy catheter having a distally extending cutting element according to another embodiment of the present invention.

FIGS. 5-7 are schematic sectional view illustrations of different embodiments for attaching a distally extending cutting element to the distal end of a hydrodynamic thrombectomy catheter.

FIGS. 8-9 diagrammatically illustrate the steps of a method of removing a blood clot located within a body lumen of a blood vessel.

FIGS. 10A-10B are schematic sectional view illustrations of a dual lumen hydrodynamic thrombectomy catheter according to another embodiment of the present invention.

FIG. 11 is a schematic sectional view illustration of a distal portion of a hydrodynamic thrombectomy catheter according to another embodiment of the present invention.

FIG. 12 is a schematic sectional view illustration of a distal portion of a hydrodynamic thrombectomy catheter according to an embodiment of the present invention, wherein the catheter includes multiple distally extending cutting elements.

FIGS. 13-14 are schematic sectional view illustrations of a hydrodynamic thrombectomy catheter according to another embodiment of the present invention.

FIG. 15 is a schematic sectional view illustration of a distal portion of a hydrodynamic thrombectomy catheter having a centering balloon according to another embodiment of the present invention.

FIG. 16 is an illustration of a motor system suitable for use with a hydrodynamic thrombectomy catheter according to an embodiment of the present invention.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician. The term “hydrodynamic” is used in the following description with respect to being related to or operated by the force of a liquid in motion.

The following detailed description is merely exemplary in nature and is not intended to limit the invention or the application and uses of the invention. Although the description of the invention is in the context of treatment of blood vessels such as the cranial, coronary, carotid and renal arteries, the invention may also be used in any other body passageways where it is deemed useful. Furthermore, there is no intention to be bound by any expressed or implied theory presented in the preceding technical field, background, brief summary or the following detailed description.

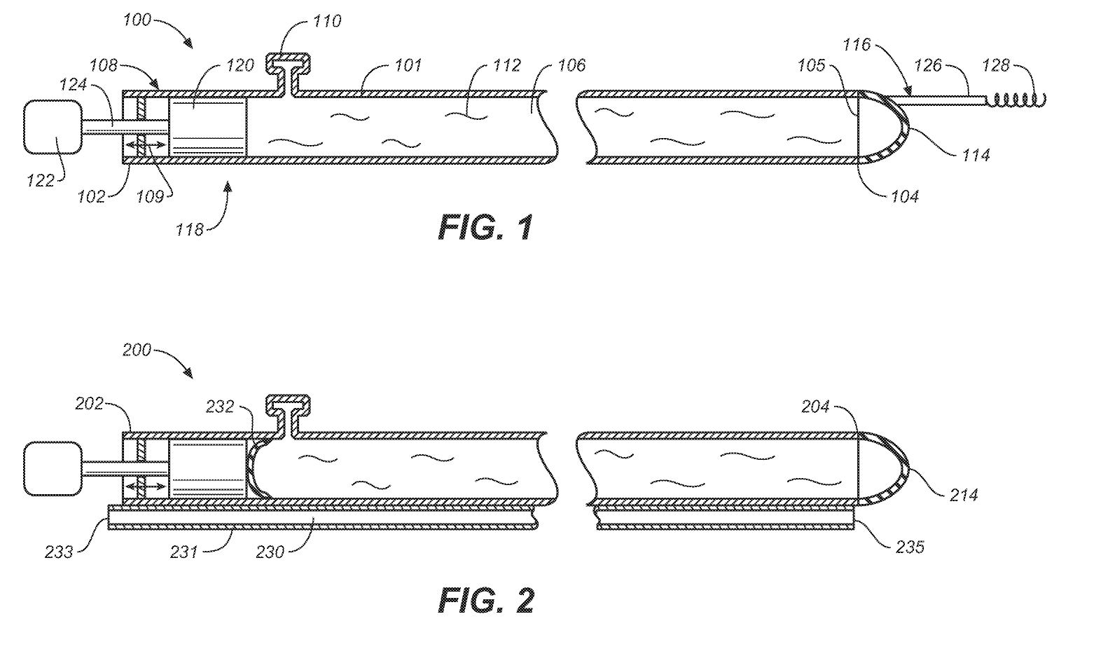

Embodiments of the present invention are directed to a thrombectomy catheter device that uses hydrodynamic fluid flow to transfer energy from a proximal end to a distal end of the catheter, thereby creating an oscillating distal tip that mechanically breaks up a blood clot. Since the device does not require a stiff and inflexible drive shaft extending the length of the catheter, the device is flexible and has a lower profile in order to access very small and tortuous vessels such as those in the intracranial circulation. Further details and description of embodiments are provided below with reference to FIGS. 1-16.

FIG. 1 illustrates a schematic sectional view of a hydrodynamic thrombectomy catheter 100 for removing an obstruction within a body lumen. Catheter 100 includes a catheter shaft 101, which is an elongate tubular shaft defining a lumen 106 extending from a proximal end 102 to a distal end 104 of catheter shaft 101. Proximal end 102 of catheter shaft 101 extends out of the patient and may be manipulated by a clinician, and distal end 104 of catheter shaft 101 is positionable at a target location within the vasculature. A flexible membrane 114 is attached to catheter shaft 101 over distal end 104 such that membrane 114 fluidly seals a distal port 105 of distal end 104. During operation, a volume of fluid 112 is contained within lumen 106 of catheter shaft 101 by membrane 114. Fluid 112 may be a contrast solution, as used in the art for flushing catheters and inflating balloons for visualization under fluoroscopy. For example, fluid 112 may be CONRAY Iothalamate Meglumine Injection USP 60% mixed 1:1 with saline. As indicated by directional arrow 109, an actuating mechanism 118 is operatively connected to proximal end 102 of catheter shaft 101 to move the working volume of fluid 112 in such a manner that fluid 112 causes membrane 114 to cyclically expand and contract, or as otherwise stated, to oscillate. A cutting element or tool 116 is attached to and extends distally from flexible membrane 114, such that oscillations of membrane 114 also cause oscillating deflections of cutting element 116. Accordingly, hydrodynamic thrombectomy catheter 100 uses pulsatile fluid flow through catheter shaft 101 to transmit energy from actuating mechanism 118 at the proximal end of the catheter to membrane 114 at distal end 104 of the catheter. The transmitted energy causes cutting element 116 to oscillate and cut through or macerate a target blood clot.

Catheter shaft 101 is a long flexible tubular shaft made of any suitable material. The catheter may have any suitable working length, for example, 50 cm-200 cm, in order to extend to a target location within the vasculature. Non-exhaustive examples of polymeric materials for catheter shaft 101 are HDPE, PEEK, PEBAX, polyethylene terephalate (PET), nylon, silicone, polyethylene, LDPE, HMWPE, polyurethane, or combinations of any of these, either blended or co-extruded. In one embodiment, the entire catheter shaft may be formed from a metallic material such as stainless steel or nitinol. In one embodiment, a proximal portion of the catheter may be formed of a metallic material, such as stainless steel or nitinol, or as a composite having a reinforcement material incorporated within a polymeric body in order to enhance strength, flexibility, and/or toughness. Suitable reinforcement layers include braiding, wire mesh layers, embedded axial wires, embedded helical or circumferential wires, and the like. In an embodiment, the proximal portion of the catheter may in some instances be formed from a reinforced polymeric tube, for example, as shown and described in U.S. Pat. No. 5,827,242 to Follmer et al. which is incorporated by reference herein in its entirety.

Catheter 100 includes a port 110 located near proximal end 102 of catheter shaft 101. Port 110 is in fluid communication with lumen 106, and is utilized for adding fluid 112 to catheter shaft 101 by the operator. Port 110 may also be utilized for aspirating or removing air from lumen 106 via a syringe, or other suitable device, prior to adding fluid 112 thereto. It may not always be desirable to add fluid 112 until distal end 104 of catheter shaft 101 is tracked to and positioned adjacent to the target location within the vasculature because catheter shaft 101 may be more flexible prior to the addition of fluid 112. However, once fluid 112 is added via port 110, port 110 is sealed in order to create a working volume of fluid within catheter 100.

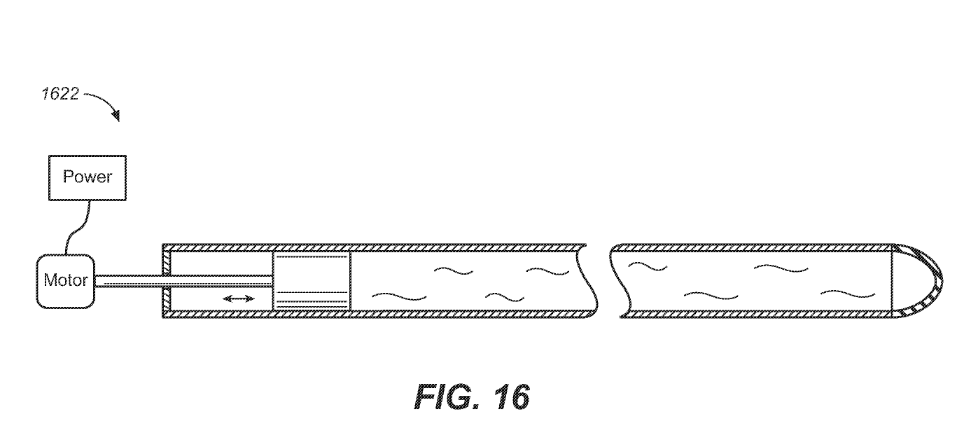

Actuating mechanism 118 provides a force to cyclically displace or move fluid 112 contained within lumen 106 of catheter shaft 101. Actuating mechanism 118 may be at least partially housed within a proximal portion 108 of catheter shaft 101. In one embodiment, actuating mechanism 118 includes a piston 120 that is slidable within catheter shaft 101 and is in contact with fluid 112. Piston 120 is a disk or cylindrical member tightly fitting and moving within lumen 106 of catheter shaft 101, and is operative to displace or move fluid 112. As illustrated in FIG. 2, the actuating mechanism may alternatively include a distendable diaphragm 232 in contact with fluid 112 inside of catheter 100 rather than a piston for displacing or moving fluid 112. With no forces acting thereto, piston 120 is contained within catheter shaft proximal portion 108 and connected through a linkage member 124 to a motor 122. Motor 122 may be contained within catheter shaft proximal portion 108 or may be external to the device, and in one embodiment, may be powered by a battery. When motor 122 is activated, linkage member 124 acts to push and pull piston 120, thus displacing fluid 112 within catheter shaft 101. The frequency of the input oscillations is adjustable depending on the particular application. In one embodiment, the frequency of the input oscillation is between approximately 40 Hz to 250 Hz. However, it should be noted that there is no upper limit for the frequency of input oscillations. One example of a suitable motor system is shown in FIG. 16. Motor system 1622 includes a power source and a motor that generates an oscillating motion to drive the piston back and forth. Any suitable motor may be utilized as long as the motor generates sufficient fluid pressure to oscillate the membrane and/or cutting member at the distal end of the catheter. In another embodiment (not shown), the actuating mechanism may alternatively include a push-pull syringe for providing the driving force that will cyclically displace or move fluid 112.

In one embodiment, flexible membrane 114 is a diaphragm or distendable dome-shaped member attached to distal end 104 of catheter shaft 101 such that membrane 114 fluidly seals distal port 105. As will be described in more detail below, flexible membrane 114 is not limited to a dome-shaped member but may have alternative configurations, such as a preformed balloon or a cylinder having one closed end. When motor 122 is activated, displacement of fluid 112 within catheter shaft 101 is translated to flexible membrane 114, thus causing it to expand radially and/or longitudinally. If cutting element 116 is present, cutting element 116 also moves radially and/or longitudinally. As piston 120 is proximally retracted to its proximalmost position, membrane 114 in turn returns to its relaxed or unexpanded position. The cyclic operation of catheter 100 allows membrane 114 and cutting element 116 to oscillate at a controlled rate in order to break up or macerate a target blood clot. It should be noted that neither flexible membrane 114 nor cutting element 116 is required to come into contact with the vessel wall to be effective. Rather, expansion of the flexible membrane 114 causes cutting element 116 to oscillate within a target blood clot.

In another embodiment illustrated in FIG. 2, the thrombectomy catheter may include an aspiration lumen to remove the debris created by the thrombectomy catheter and prevent the release of thrombotic or embolic particles into the bloodstream during the procedure. More particularly, thrombectomy catheter 200 includes an elongate tubular aspiration shaft 231 extending from a proximal end 202 to a distal end 204 of catheter 200. Aspiration shaft 231 defines an open aspiration lumen 230, which extends the full length of catheter 200. In the embodiment depicted in FIG. 2, aspiration lumen 230 is formed by attaching an additional shaft to a single-lumen catheter. Alternatively, as understood by those of ordinary skill in the art, catheter 200 may be formed using an extruded tubular shaft having dual lumens extending in parallel or side-by-side along the full length thereof (not shown). Dual-lumen profile extrusions may have parallel round lumens surrounded by relatively uniform walls, resulting in a non-circular, generally figure-eight shaped transverse cross-section. Alternatively, if a circular outer profile is desired, then dual-lumen profile extrusions may have parallel round lumens with non-uniform wall thicknesses or various other combinations of lumens having unequal sizes and non-round cross-sectional shapes such as D-shapes or crescent-shapes, as would be understood by one of skill in the art. Aspiration lumen 230 fluidly connects a proximal port 233 disposed at or adjacent the proximal end of aspiration shaft 231 with a distal port 235 disposed at or adjacent the distal end of aspiration shaft 231. When aspiration lumen 230 is to be activated, a source (not shown) of partial vacuum or “negative pressure” may be connected to the luer adaptor of a fitting (not shown) mounted at the proximal end of aspiration shaft 231 in fluid communication with proximal port 233 in order to aspirate blood and particulates through aspiration lumen 230 of catheter 200.

In addition to removing debris created by the thrombectomy catheter, aspiration lumen 230 may also serve as a guidewire lumen such that catheter 200 may be tracked over a guidewire when being delivered to the treatment site. In such an embodiment, lumen 230 would be at least of a sufficient diameter to slidingly accept a medical guidewire therethrough. Once catheter 200 is tracked to the target site over a guidewire, the guidewire may be retracted and removed in order to allow aspiration lumen 230 to capture relatively large thrombotic or embolic particles. In one embodiment the size of the aspiration lumen and guidewire, relative to each other, are such that the guidewire does not have to be withdrawn for aspiration to occur.

Although not shown in every figure, it will be apparent to those skilled in the art that the use of an aspiration shaft and lumen may be utilized with any embodiment described herein. However, an aspiration lumen is not required for removing debris created by the thrombectomy catheter. In one embodiment, operation of the thrombectomy catheter breaks up the target blood clot into small enough pieces that the debris is allowed to migrate downstream once normal blood flow is re-established. The broken-up pieces of the clot are sufficiently small that they will not get lodged at a point within the vasculature where they would cause a significant problem. In another embodiment, the thrombectomy catheter may be operated in conjunction with an infused thrombolytic agent that dissolves the separated thrombotic or embolic particles to small enough dimensions that they may be released into the bloodstream during the procedure. As such, the clot is broken down by both pharmaceutical and mechanical mechanisms. Non-exhaustive examples of suitable thrombolytic agents include tissue plasminogen activator (t-PA), or urokinase.

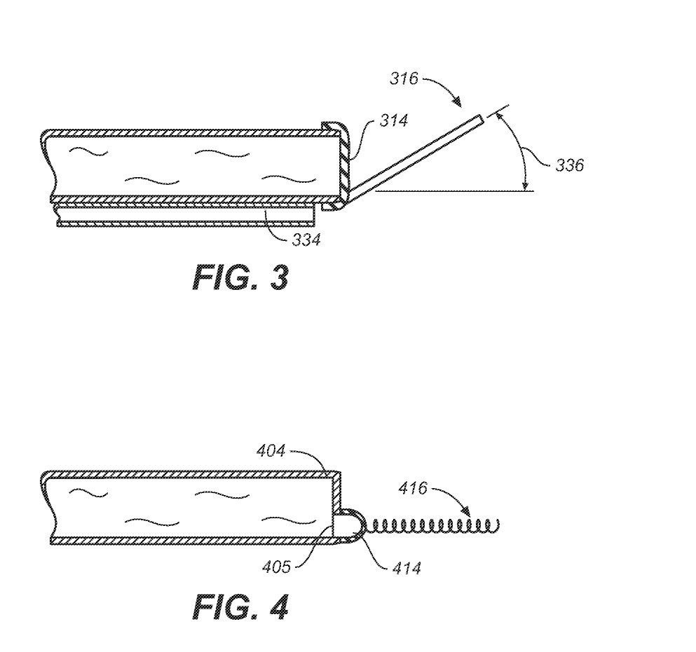

Referring now to FIG. 3, the thrombectomy catheter may be of the so-called single operator or rapid-exchange type. The thrombectomy catheter includes a substantially shorter guidewire shaft 334 defining a guidewire lumen extending along a distalmost portion of the catheter. As such, the guidewire is located outside of the thrombectomy catheter except for a short guidewire segment that extends within the guidewire lumen. Advantageously, a clinician is able to access portions of the guidewire proximal and distal of guidewire shaft 334 while the thrombectomy catheter is loaded or exchanged onto the guidewire, which may be already indwelling in the patient. The thrombectomy catheter is then advanced through the patient's vasculature with only a distal portion of the catheter riding along the guidewire.

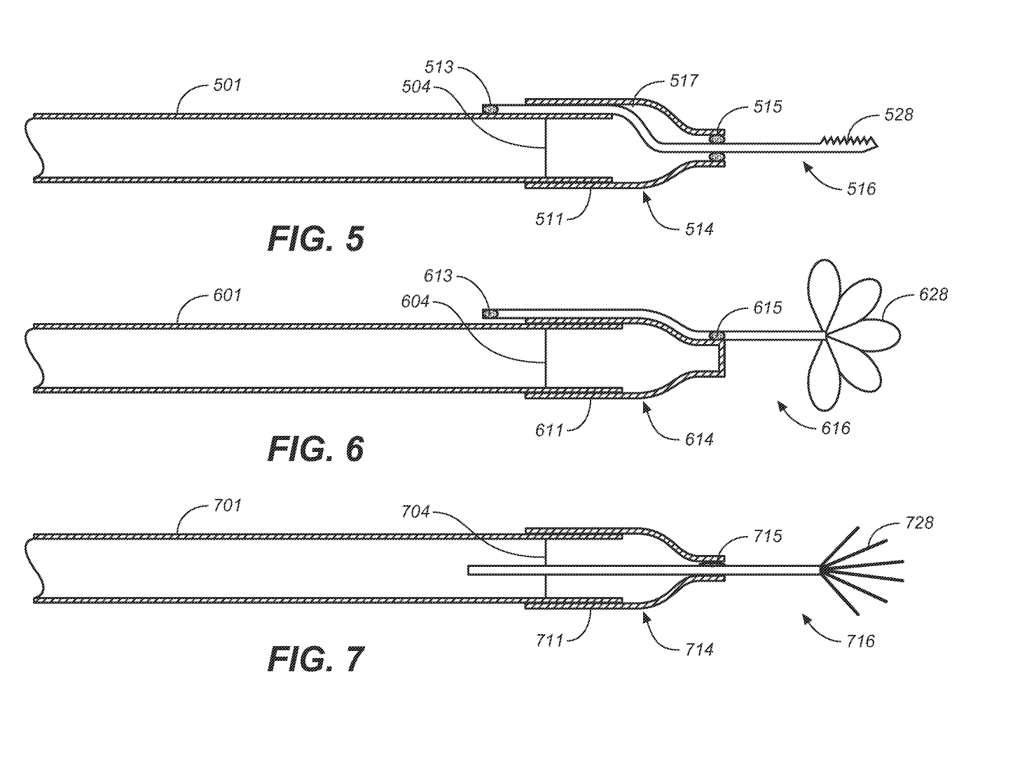

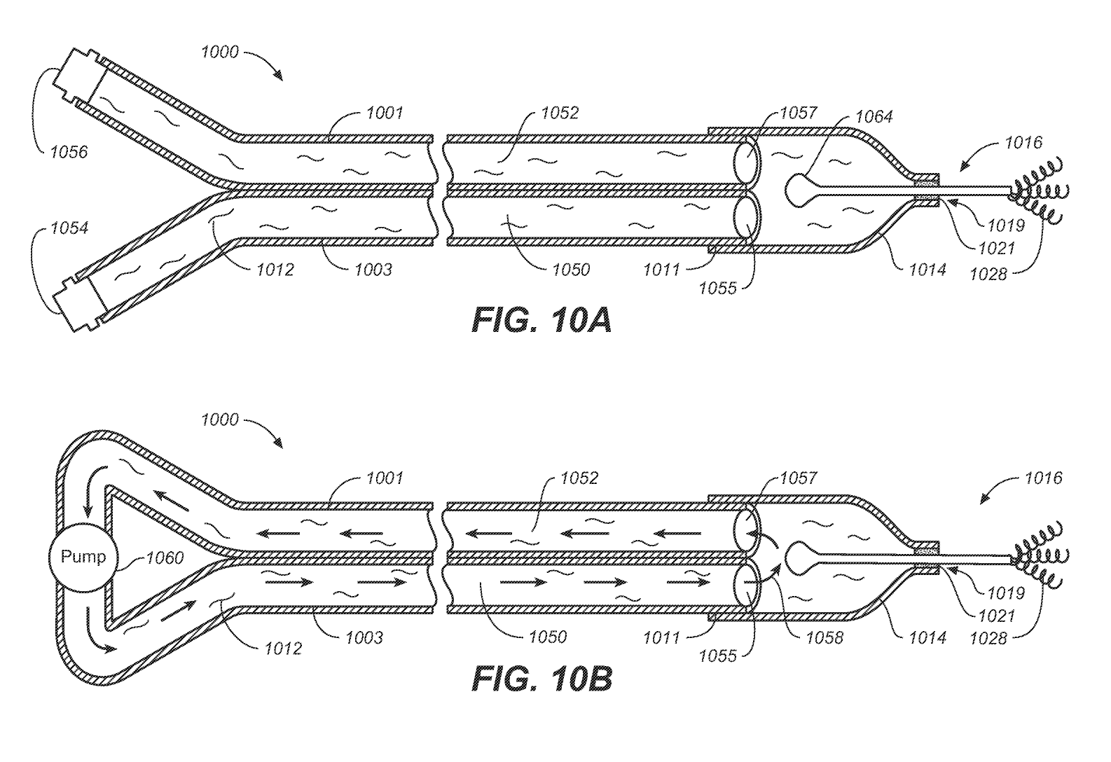

As previously described, embodiments of the hydrodynamic thrombectomy catheter use pulsatile fluid flow to transmit energy through the catheter shaft from the driving mechanism at the proximal end of the catheter to the flexible membrane at the distal end of the catheter. In one embodiment illustrated in FIG. 2, the transmitted energy causes flexible membrane 214 to rapidly expand and contract, thereby mechanically breaking up or pulverizing a target blood clot into smaller pieces. In other embodiments of the present invention, one or more cutting elements may be attached to and distally extend from the flexible membrane, such that oscillations of the flexible membrane also cause oscillating deflections of the cutting element(s). For example, the cutting element may be formed from a floppy guidewire tip or other small flexible wire or polymer fiber that will oscillate side to side and/or back and forth during expansion of the flexible membrane. The wire or polymeric fiber may have any suitable cross-section, including but not limited to a circular, rectangular, square, or elliptical cross-section. The cutting element may have any configuration suitable for breaking up a clot. Referring to FIG. 1, in one embodiment, cutting element 116 includes a portion of a guidewire having a straight portion 126 and a coiled or curly portion 128 at the distal end thereof. Alternatively, a cutting element 316 may be a straight flexible wire or polymeric fiber as shown in FIG. 3 or a cutting element 416 may be continuously coiled along the length thereof as shown in FIG. 4. The cutting element may extend parallel to a longitudinal axis of the device, as shown in FIG. 1, or may extend from the flexible membrane at an angle with respect to the longitudinal axis of the device. For example, as shown in FIG. 3, cutting element 316 extends from flexible membrane 314 at an angle 336. When oscillating, angle 336 increases radial displacement of cutting element 316 and results in a random, whipping motion of cutting element 316. Other suitable configurations for the cutting element are illustrated in FIGS. 5-7 and 10A-10B, including a cutting element 516 having a saw-tooth portion 528 as shown in FIG. 5, a cutting element 616 having multiple distally-extending loops 628 as shown in FIG. 6, a cutting element 716 having multiple distally-extending straight members 728 as shown in FIG. 7, and a cutting element 1016 having multiple distally-extending coils 1028 as shown in FIGS. 10A-10B.

The flexible membrane may be formed from various materials and may have various configurations. For example, the flexible membrane may be constructed from an elastomeric material requiring a low inflation pressure to expand, or may be formed from a non-elastomeric thin walled polymer requiring a slightly higher inflation pressure to expand. The flexible membrane may be secured to the distal end of the catheter shaft via a suitable mechanical method, such as via an adhesive, a solvent bond, thermal bonding, and/or an over sleeve. In one embodiment, the flexible membrane may be a segment or piece of material covering the distal port of the catheter shaft, resulting in a dome-shape unexpanded configuration such as shown in FIGS. 1-2. Alternatively, as shown in FIG. 3, membrane 314 may be a cylinder or tube of flexible material having one closed end such that when attached over the distal end of the catheter shaft, membrane 314 fluidly seals the distal port. In yet another embodiment, the flexible membrane may be a segment of a preformed balloon or a segment of tubing attached to the distal end of the catheter shaft, wherein the distal end thereof is closed resulting in a funnel-shape unexpanded configuration such as shown in FIGS. 5-7. The flexible membrane may cover or extend over the entire circumference of the catheter, as shown in FIGS. 1-3, or may cover or extend over only a portion of the circumference of the catheter as shown in FIG. 4. In FIG. 4, a distal end 404 of the catheter is partially closed to result in a smaller distal port 405. A dome-shaped flexible membrane 414 covers or extends over distal port 405 in order to fluidly seal distal port 405. A smaller flexible membrane, such as flexible membrane 414, requires less volume for expansion and thus results in increased oscillations of flexible membrane 414 and more focused displacement or deflection of cutting element 416.

A cutting element may be bonded to the flexible membrane in any suitable manner. The bond(s) may be formed with an adhesive such as DYMAX adhesive, a solvent bond, a thermal bond, or by another mechanical method, such as a heat shrinkable band. For example, FIGS. 5-7 are schematic sectional view illustrations of different embodiments for attaching a distally extending cutting element to a flexible membrane attached to the distal end of a hydrodynamic thrombectomy catheter. In FIGS. 5-7, the flexible membrane is a segment of a preformed balloon formed out of an elastomeric material, including but not limited to Elasthane, Chronothane, Tecothane, Estane, Pebax, Silicone, Urethane or a segment of C-flex tubing and the distally extending cutting element is a floppy tip guidewire having a diameter of approximately 0.014 inches. In FIG. 5, a bond 513 attaches a proximal end of cutting element 516 to the outside of catheter shaft 501 and a bond 515 attaches an intermediate portion of cutting element 516 to the inside surface of a distal end of flexible membrane 514. Cutting element 516 extends between flexible membrane 514 and the outer surface of catheter shaft 501 and curves over catheter shaft distal end 504 to extend approximately parallel with a longitudinal axis of the catheter. A proximal end 511 of flexible membrane 514 is attached to distal end 504 of catheter shaft 501, as well as to an intermediate portion of cutting element 516. In FIG. 6, cutting element 616 lies on the outside surface of flexible membrane 614. More particularly, a proximal end 611 of flexible membrane 614 is attached to distal end 604 of catheter shaft 601. A bond 613 then attaches a proximal end of cutting element 616 to the outside of catheter shaft 601 and a bond 615 attaches an intermediate portion of cutting element 616 to the outside surface of a distal end of flexible membrane 614. Lastly, in FIG. 7, cutting element 716 extends parallel with a longitudinal axis of the catheter and passes through a lumen of the flexible membrane 714. More particularly, a proximal end 711 of flexible membrane 714 is attached to distal end 704 of catheter shaft 701. A bond 715 attaches and seals an intermediate portion of cutting element 716 within a tubular opening in a distal end of flexible membrane 714.

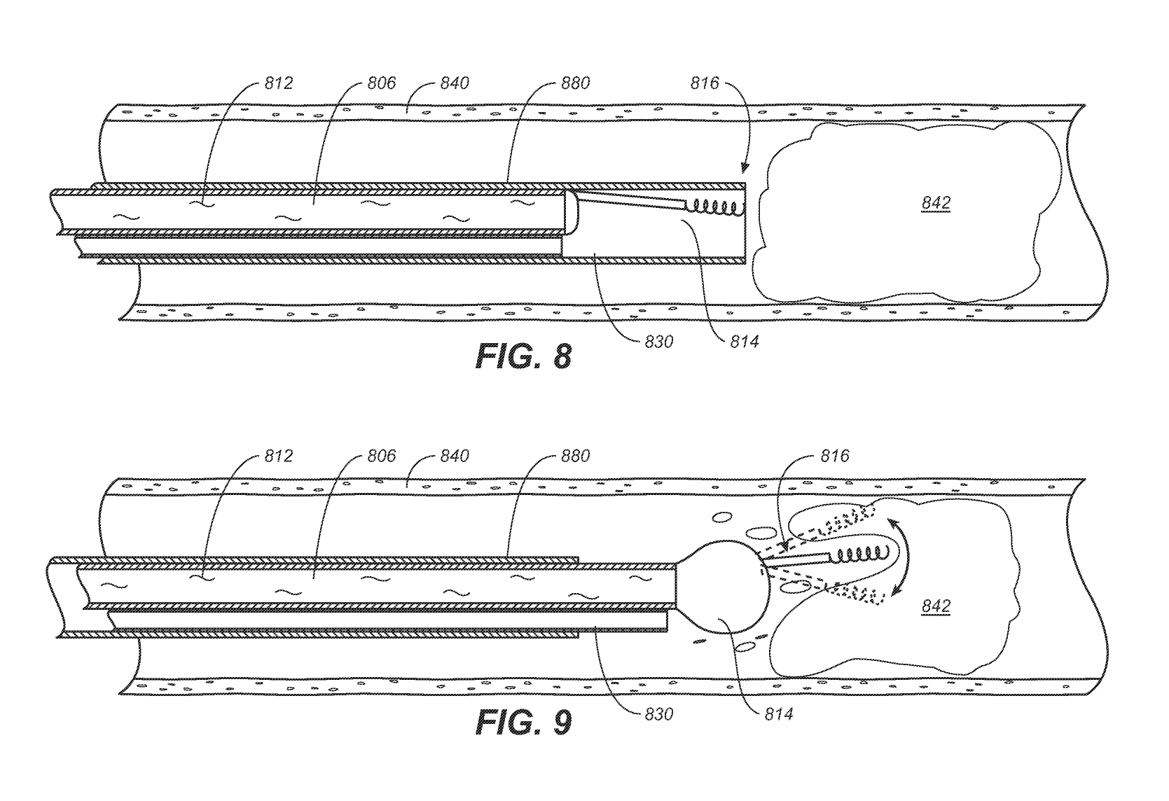

FIGS. 8-9 diagrammatically illustrate the steps of a method of removing a blood clot 842 located within a body lumen of a blood vessel 840. Typically, a guidewire is first inserted into a patient's vasculature (not shown) and advanced to the blood clot 842. As shown in FIG. 8, a hydrodynamic thrombectomy catheter according to an embodiment of the present invention is tracked over the guidewire and is positioned by a clinician such that a distally extending cutting element 816 is proximally adjacent to and partially extends within clot 842. The hydrodynamic thrombectomy catheter includes a distal flexible membrane 814 and an aspiration lumen 830. Once the catheter is properly in position, the guidewire may be retracted and removed in order to allow relatively large thrombotic or embolic particles to be removed via aspiration lumen 830. If not already completed, a clinician may aspirate or remove any air within lumen 806 via a port (not shown) at the proximal end of the catheter and then may add fluid 812 to lumen 806 of the catheter. The port is then sealed in order to create a working volume of fluid within lumen 806.

Referring now to FIG. 9, an actuating mechanism at the proximal end of the catheter is activated in order to displace or move fluid 812. As illustrated in FIG. 9, displacement of fluid 812 within catheter lumen 806 is translated to flexible membrane 814, thus causing flexible membrane 814 to expand radially and longitudinally and thereby moving cutting element 816. The actuating mechanism operates to rapidly and cyclically oscillate flexible membrane 814 and cutting element 816 at a controlled rate in order to break up or macerate blood clot 842 into smaller pieces. As blood clot 842 is macerated, aspiration lumen 830 collects the debris created by the thrombectomy catheter to prevent the release of thrombotic or embolic particles into the bloodstream during the procedure. Cutting element 816 continually oscillates and is maneuvered within clot 842 until clot 842 is mostly or entirely removed, or at least until blood flow is reestablished though vessel 840.

Although operation of the thrombectomy catheter is described in FIGS. 8-9 above with the use of a guidewire, it will be apparent to those skilled in the art that a guidewire is not necessarily required for positioning the catheter at the target blood clot. In one embodiment, the catheter shaft of the thrombectomy catheter is flexible enough to be advanced without the use of a guidewire, similar to a microcatheter. Further, as shown in FIGS. 8-9, if a cutting element is present, a flexible retractable sheath 880 may be provided to cover and protect the cutting element on the distal end of the catheter during navigation to the clot. Once the distalmost end of the catheter is located at the clot, sheath 880 may be retracted and removed to expose the cutting element. Retractable sheath 880 may be utilized for protecting a cutting element or tool at the distal end of the catheter regardless of whether a guidewire is used for positioning the catheter at the target blood clot.

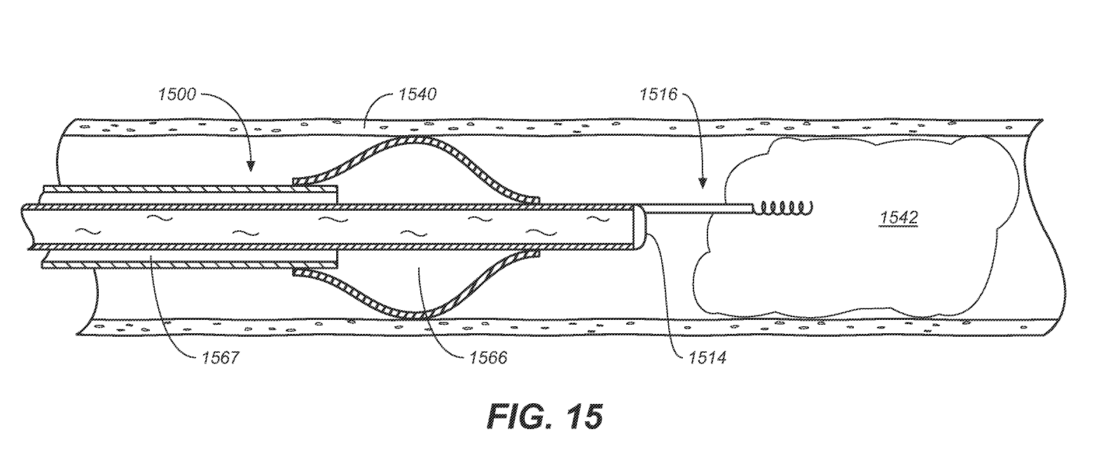

In addition, the operation of a thrombectomy catheter according to embodiments hereof may include inflation of a centering balloon to stabilize the catheter within the vessel during use. For example, FIG. 15 is a schematic illustration of a distal portion of a hydrodynamic thrombectomy catheter 1500 including a centering balloon according to another embodiment of the present invention. Catheter 1500 includes an inflatable balloon 1566 located around the distal portion of catheter 1500, proximal to flexible membrane 1514 and cutting element 1516. Balloon 1566 is inflated during operation in order to press against vessel wall 1540, thus stabilizing and centering catheter 1500 within the vessel while cutting element 1516 breaks up blood clot 1542. In addition to a lumen for containing the hydrodynamic fluid for oscillating flexible membrane 1514 and cutting element 1516 as described above, catheter 1500 also includes an inflation lumen 1567 in fluid communication with an interior of balloon 1566 to provide for inflation of balloon 1566. As understood by those of ordinary skill in the art, catheter 1500 may include a coaxial dual lumen arrangement (not shown) along the full length thereof to define the inflation lumen.

FIGS. 10A-10B are schematic sectional view illustrations of a dual lumen hydrodynamic thrombectomy catheter 1000 according to another embodiment of the present invention. Catheter 1000 includes a first catheter shaft 1001 having a first lumen 1052 extending between a proximal port 1056 and a distal port 1057, and a second catheter shaft 1003 having a second lumen 1050 extending between a proximal port 1054 and a distal port 1055, wherein the shafts 1001,1003 are secured together along a length thereof. In the embodiment depicted in FIG. 10, catheter 1000 is formed by attaching two single-lumen shafts together. Alternatively, as will be understood by those of ordinary skill in the art, catheter 1000 may be formed using an extruded tubular shaft having dual lumens extending parallel or side-by-side along the full length thereof (not shown). Dual-lumen extrusions may have parallel round lumens surrounded by relatively uniform walls, resulting in a non-circular, generally figure-eight shaped transverse cross-section. Alternatively, if a circular outer profile is desired, then dual-lumen extrusions may have parallel round lumens with non-uniform wall thicknesses or various other combinations of lumens having unequal sizes and non-round cross-sectional shapes such as D-shapes or crescent-shapes, as would be understood by one of skill in the art.

A proximal end 1011 of a membrane 1014 is bonded to the outside surfaces of shafts 1001, 1003 such that membrane 1014 fluidly seals distal ports 1055, 1057 of shafts 1003, 1001, respectively. A cutting element 1016 extends parallel with a longitudinal axis of the catheter and passes through a lumen of the membrane 1014. In this embodiment, a proximal end 1064 of cutting element 1016 extends within catheter 1000. Cutting element 1016 passes through membrane 1014. A bond 1019 attaches an intermediate portion of cutting element 1016 to the inside surface of membrane 1014. In this embodiment, bond 1019 includes a segment of tubing 1021 that is filled with adhesive and bonded to the inside surface of a distal end of membrane 1014 in order to secure cutting element 1016 and also operate as a weight. As cyclic fluid flow passes against proximal end 1064 of cutting element 1016, the fluid flow causes cutting element 1016 to move in a pulsative manner. Bond 1019 acts as a hinge or pivot point as the multiple distally-extending coils 1028 of cutting element 1016 oscillate side to side and/or back and forth. As compared to the remaining length thereof, proximal end 1064 of cutting element 1016 may have an increased surface area, such as a paddle shape shown in FIGS. 10A-10B, to enhance the movement of cutting element 1016. In an embodiment where the movement of proximal end 1064 causes the movement of cutting element 1016, membrane 1014 is not required to oscillate and thus need not be of a flexible material. Rather, membrane 1014 may be formed from any suitable material that fluidly seals distal ports 1055, 1057 of catheter 1000. However, when formed from a flexible material, rapid and cyclic oscillations of membrane 1014 will enhance the movement of cutting element 1016.

In the embodiment depicted in FIG. 10A, an actuating mechanism, such as one including a motor, motor linkage, and piston or diaphragm as described above, may be located at proximal port 1054 of catheter shaft 1003. Fluid 1012 may be added via proximal port 1056 of catheter shaft 1001 and then sealed to create a working volume of fluid within catheter 1000. Similar to the embodiments described above, the actuating mechanism is activated in order to displace or move fluid 1012. A force from the actuating mechanism is translated to paddle-like proximal end 1064 and/or membrane 1014 to cause coils 1028 of cutting element 1016 to rapidly and cyclically oscillate at a controlled rate in order to break up or macerate a target blood clot. Although the actuating mechanism is described as located at port 1054 with port 1056 being described as sealed, it will be apparent to those of ordinary skill in the art that the actuating mechanism may alternatively be located at port 1056 and port 1054 may be sealed. In another embodiment (not shown), the actuating mechanism may include a dual syringe system in which a first syringe (not shown) is located at proximal port 1054 to push/pull fluid 1012 through catheter shaft 1003 and a second syringe (not shown) is located proximal port 1056 to pull/push fluid 1012 proximally through catheter shaft 1001.

Alternatively, in the embodiment depicted in FIG. 10B, the actuating mechanism may include a peristaltic pump 1060 connected between catheter shafts 1001, 1003 to create a closed circuit or system. Peristaltic pump 1060 acts to circulate a working volume of fluid 1012 through the lumens of catheter 1000. More particularly, when activated, peristaltic pump 1060 creates pulsatile flow by cyclically pushing fluid 1012 through lumen 1050 of catheter shaft 1003 and pulling fluid 1012 through lumen 1052 of catheter shaft 1001. As such fluid 1012 is circulated through lumens 1050, 1052, as indicated by directional arrows 1058, in a manner that rapidly and cyclically oscillates paddle-like proximal end 1064 and/or membrane 1014 to cause coils 1028 of cutting element 1016 to break up or macerate a target blood clot. Peristaltic pump 1060 is a type of positive displacement pump used for pumping a fluid contained within a flexible tube fitted inside the pump casing. Peristaltic pumps are typically used in medical applications to pump clean or sterile fluids because the pumping mechanism does not contact and therefore cannot contaminate the fluid. A rotor with a number of rollers, shoes or wipers attached to the external circumference compresses the flexible tube as the rotor turns, such that the part of the tube under compression closes, or occludes, thus forcing the fluid to move through the tube. Additionally, the tube opens to its natural state after the passing of the cam, aka, restitution, and fluid flow is induced into the pump. In one embodiment, a suitable pump 1060 includes a compact, 12 volt direct current water pump having a high pressure capacity.

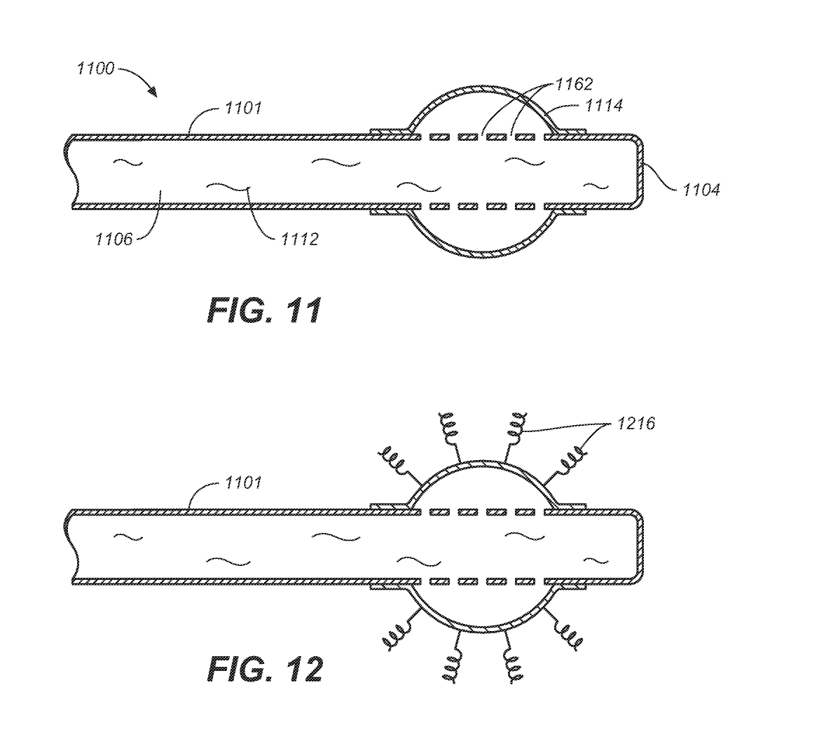

FIGS. 11-12 are schematic sectional view illustrations of a distal portion of a hydrodynamic thrombectomy catheter 1100 according to another embodiment of the present invention. In FIGS. 11-12, flexible membrane 1114 is mounted around the outside surface of a catheter shaft 1101 rather than mounted over a distal port of a catheter shaft as in previous embodiments. Catheter shaft 1101 is a single lumen tubular shaft having a closed distal end 1104 in order to contain a working volume of fluid 1112 within a lumen 1106 of catheter 1100. Lumen 1106 is in fluid communication with the interior volume of flexible membrane 1114 via holes or ports 1162 formed into catheter shaft 1101 to enable expansion and subsequent oscillations of flexible membrane 1114 when fluid 1112 is displaced by a actuating mechanism (not shown) located at a proximal end of the catheter 1100. As shown in FIG. 12, a plurality of cutting elements 1216 may be attached to flexible membrane 1114 such that oscillations of flexible membrane 1114 also cause oscillations of cutting elements 1216 to assist in breaking up a target blood clot.

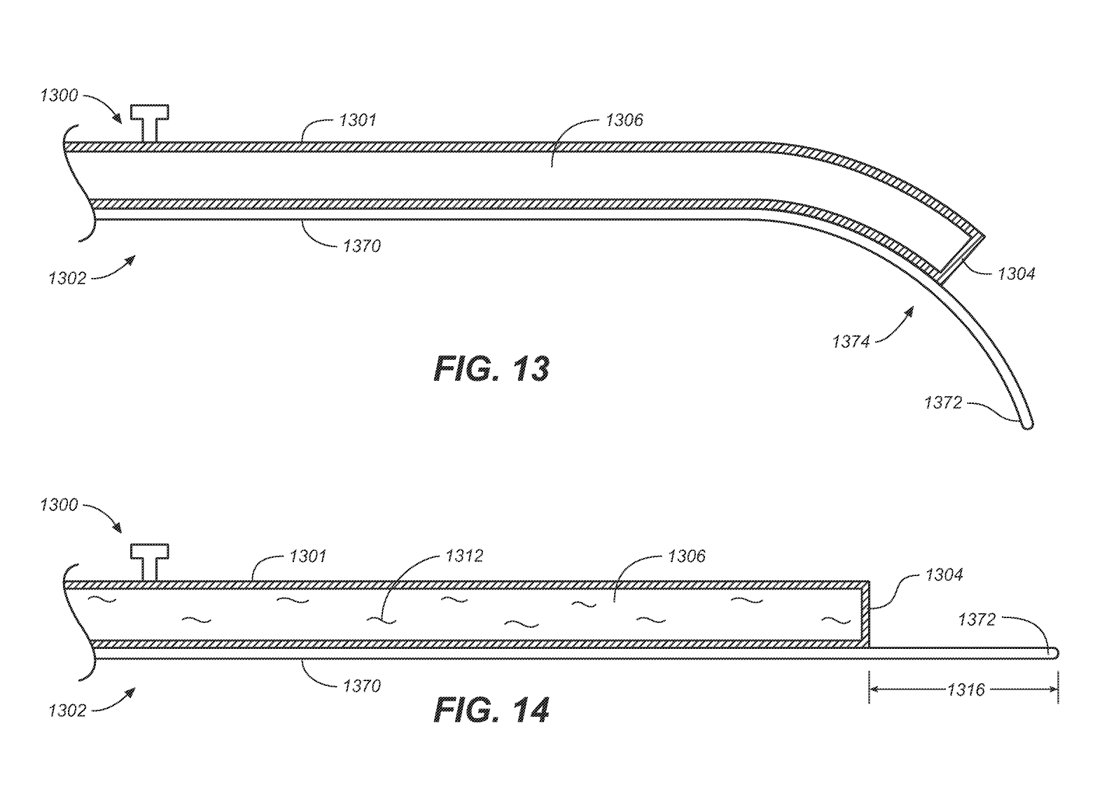

FIGS. 13-14 are schematic sectional illustrations of a hydrodynamic thrombectomy catheter 1300 according to another embodiment of the present invention. Catheter 1300 includes a catheter shaft 1301 and a wire member 1370 attached thereto. Catheter shaft 1301 is an elongate flexible tube defining a lumen 1306 extending from a proximal end 1302 to a sealed or closed distal end 1304 of catheter shaft 1301. It should be noted that any structure or configuration may be utilized for closing or sealing distal end 1304 of catheter shaft 1301. For example, a cylindrical stopper may be inserted into the distal port of lumen 1306 or a membrane structure, as noted in the embodiments above, may be attached over distal end 1304 to fluidly seal the distal port of lumen 1306. In another embodiment, the distal port of lumen 1306 may be heat treated or undergo another manufacturing process to fluidly seal and close distal end 1304 of catheter shaft 1301.

Wire member 1370 is attached to catheter shaft 1301, and extends at least along the distal portion of catheter shaft 1301. In one embodiment, wire member 1370 extends along the entire length thereof as shown in FIG. 13. Wire member 1370 is attached or secured to catheter shaft 1301 via a suitable mechanical method, such as via an adhesive, a solvent bond, thermal bonding, and/or an over sleeve. Wire member 1370 may be attached along an outside surface of catheter shaft 1301 as shown in FIGS. 13-14, but may alternatively be attached along an inside surface of catheter shaft 1301 (not shown) or extend through the wall of catheter shaft 1301 (not shown). Wire member 1370 is formed from a shape memory material such as nickel-titanium (nitinol) and includes a bend 1374 along the distal length thereof as shown in FIG. 13. Shape memory metals are a group of metallic compositions that have the ability to return to a defined shape or size when subjected to certain stress conditions.

A distal end 1372 of wire member 1370 extends distally beyond distal end 1304 of catheter shaft 1301 to define cutting element 1316 of catheter 1300. During operation, cutting element 1316 oscillates and cuts through or macerates a target blood clot as will be described in more detail below. As explained in the previously described embodiments, cutting element 1316 may have any configuration suitable for breaking up a clot. In FIGS. 13 and 14, cutting element 1316 is defined by the distalmost length of wire member 1370 that extends beyond catheter shaft distal end 1304 and is shown as a straight cutting element similar to that of FIG. 3. Alternatively, the distalmost length of wire member 1370 may be shaped or formed into a coiled or curly configuration to resemble the cutting elements described above in relation to the embodiments of FIG. 1 or 4. In various other embodiments, a separate cutting element as illustrated in FIGS. 5-7 and 10A-10B may be attached at distal end 1372 of wire member 1370, such that a cutting element having a saw-tooth portion as shown in FIG. 5, a cutting element having multiple distally-extending loops as shown in FIG. 6, a cutting element having multiple distally-extending straight members as shown in FIG. 7, and a cutting element having multiple distally-extending coils as shown in FIGS. 10A-10B may be utilized. As such, the attached cutting element may be formed from a material different from wire member 1370, such as a floppy guidewire tip or other small flexible wire or polymer fiber that will oscillate side to side and/or back and forth during operation of the hydrodynamic catheter.

During operation, a volume of fluid 1312 is contained within lumen 1306 of catheter shaft 1301 and acts in a hydrodynamic manner similar to the embodiments described above. An actuating mechanism such as a motor or syringe as described above exerts a sufficient pressure force onto fluid 1312 to straighten catheter shaft 1301, and simultaneously straighten wire member 1370 such that bend 1374 is removed as shown in FIG. 14. Stated another way, the applied pressure force is strong enough to overcome the bias of the shape memory wire member 1370. When the pressure force is removed, wire member 1370 will bias to its original bent position of FIG. 13. Accordingly, when force is applied to and removed from fluid 1312 in rapid succession, wire member 1370 will alternate between a bent configuration and a straightened configuration thereby oscillating cutting member 1316 to break-up a target blood clot. In other words, cutting element 1316 oscillates back and forth and/or side to side as wire member 1370 alternates between the bent and straightened configurations when the actuating mechanism cycles the force in rapid succession.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A catheter apparatus for treating an obstruction within a body lumen, the catheter apparatus comprising:
an elongate tubular shaft defining a lumen and having a closed distal end;
a volume of fluid contained within the lumen of the tubular shaft;
a wire member attached to the tubular shaft, the wire member having a bend occurring along a distal length thereof, wherein a distal end of the wire member extends distally beyond the distal end of the tubular shaft to define a cutting element; and
an actuating mechanism operatively connected to a proximal end of the tubular shaft that applies a sufficient force to displace the fluid disposed within the lumen of the tubular shaft to straighten the bend in the wire member, wherein the cutting element oscillates as the wire member alternates between bent and straightened configurations when the actuating mechanism cycles the force in rapid succession.

2. The catheter apparatus of claim 1, wherein the wire member is formed from a shape memory material.

3. The catheter apparatus of claim 2, wherein the shape memory material is nickel-titanium.

4. The catheter apparatus of claim 1, wherein the distal end of the tubular shaft is closed by a membrane structure.

5. The catheter apparatus of claim 1, wherein the wire member is attached along an outside surface of the tubular shaft.

6. The catheter apparatus of claim 1, wherein the wire member extends along an entire length of the tubular shaft.

7. The catheter apparatus of claim 1, wherein the distal end of the wire member forms a straight cutting element.

8. The catheter apparatus of claim 1, wherein at least a portion of the cutting element is a separate structure attached to and distally extending from the distal end of the wire member.

9. The catheter apparatus of claim 1, wherein the cutting element of the wire member includes a coiled distal end.

10. The catheter apparatus of claim 1, wherein the cutting element of the wire member includes at least one looped flexible member at a distal end thereof.

11. The catheter apparatus of claim 1, wherein the actuating mechanism includes a motor.

12. The catheter apparatus of claim 1, wherein the actuating mechanism includes a piston slidingly disposed within the lumen of the tubular shaft at the proximal end of the catheter, wherein the piston is in contact with the fluid and is operable to displace the fluid.

13. A catheter apparatus for treating an obstruction within a body lumen, the catheter apparatus comprising:
an elongate tubular shaft defining a first lumen and having a closed distal end;
a volume of fluid contained within the first lumen of the tubular shaft;
a second open-ended lumen extending side-by-side and parallel to the first lumen from a proximal end to the distal end of the tubular shaft;
a wire member formed from a shape memory material attached to the tubular shaft, the wire member having a bend occurring along a distal length thereof, wherein a distal end of the wire member extends distally beyond the distal end of the tubular shaft to define a cutting element; and
an actuating mechanism operatively connected to the proximal end of the tubular shaft that applies a sufficient force to displace the fluid disposed within the lumen of the tubular shaft to straighten the bend in the wire member, wherein the cutting element oscillates as the wire member alternates between bent and straightened configurations when the actuating mechanism cycles the force in rapid succession.

14. The catheter apparatus of claim 13, wherein the distal end of the wire member forms a straight cutting element.

15. The catheter apparatus of claim 13, wherein at least a portion of the cutting element is a separate structure attached to and distally extending from the distal end of the wire member.

16. The catheter apparatus of claim 13, wherein the cutting element of the wire member includes a coiled distal end.

17. The catheter apparatus of claim 13, wherein the cutting element of the wire member includes at least one looped flexible member at a distal end thereof.

18. A method of treating an obstruction within a body lumen, the method comprising the steps of:
positioning a catheter apparatus near the obstruction within the body lumen, wherein the catheter apparatus includes an elongate tubular shaft defining a lumen and having a closed distal end, a volume of fluid contained within the lumen of the tubular shaft, a wire member attached to the tubular shaft, the wire member having a bend occurring along a distal length thereof, wherein a distal end of the wire member extends distally beyond the distal end of the tubular shaft to define a cutting element, and an actuating mechanism operatively connected to the proximal end of the tubular shaft;
activating the actuating mechanism to displace the fluid disposed within the lumen of the tubular shaft to straighten the bend in the wire member;
cyclically displacing the fluid disposed within the lumen of the tubular shaft to cause the cutting element to oscillate as the wire member alternates between bent and straightened configurations; and
macerating at least a portion of the obstruction by the oscillating wire member.

19. The catheter apparatus of claim 18, wherein the wire member is formed from a shape memory material.

20. The method of claim 18, further comprising:
aspirating debris created by the catheter apparatus.
